(19) United States
(12) Patent Application Publication
(10) Pub. No.: US 2014/0163671 A1
(43) Pub. Date: Jun. 12, 2014

(54) LEAFLET AND VALVE APPARATUS

(71) Applicant: W. L. Gore & Associates, Inc., Newark, DE (US)

(72) Inventors: William C. Bruchman, Camp Verde, AZ (US); Cody L. Hartman, Flagstaff, AZ (US)

(73) Assignee: W. L. Gore & Associates, Inc., Newark, DE (US)

(21) Appl. No.: 14/183,251

(22) Filed: Feb. 18, 2014

Related U.S. Application Data
(63) Continuation-in-part of application No. 13/485,823, filed on May 31, 2012.
Application No. 13/485,823 is a continuation-in-part of application No. 13/078,774, filed on Apr. 1, 2011.
Application No. 13/078,774 is a continuation-in-part of application No. 13/078,774, filed on Apr. 1, 2011.
(60) Provisional application No. 61/800,402, filed on Mar. 15, 2013.

(51) Int. Cl. A61F 2/24

(52) U.S. Cl. CPC A61F 2/2412
USPC 623/2.17; 623/2.12

(57) ABSTRACT

The present invention provides a leaflet for use in a prosthetic valve that stabilizes the motion of the leaflet as it moves between a closed position and an open position. In accordance with embodiments, a prosthetic valve is provided with a leaflet that contains a stiffening element between layers of film from which the leaflet is made.

### FIELD

The present invention relates generally to valve leaflets and apparatus and systems having valve leaflets, such as prosthetic valves and more specifically, prosthetic cardiac valves.

### BACKGROUND

Bioprosthetic valves have been developed that attempt to mimic the function and performance of a native valve. Flexible leaflets are fabricated from biological tissue such as bovine pericardium. In some valve designs the biological tissue is sewn onto a relatively rigid frame that supports the leaflets and provides dimensional stability when implanted. Although bioprosthetic valves can provide excellent hemodynamic and biomechanical performance in the short term, they are prone to calcification and cusp tears, among other failure modes, requiring reoperation and replacement.

Attempts have been made to use synthetic materials, such as polyurethane, among others, as a substitute for the biological tissue, to provide a more durable flexible leaflet prosthetic valve, herein referred to as a synthetic leaflet valve (SLV). However, synthetic leaflet valves have not become a valid valve replacement option since they suffer premature failure, due to, among other things, suboptimal design and lack of a durable synthetic material.

A number of fabrication techniques have been used to couple the leaflets to a frame, including sewing individual leaflets to the frame (biological and synthetic), and for synthetic leaflets only, injection molding and dip coating a polymer onto the frame. In many cases, the resulting leaflet is supported on the frame and defines a flap having a mounting edge where the leaflet is coupled to the frame and a free edge that allows the flap to move. The flap moves under the influence of fluid pressure. In operation, the leaflets open when the upstream fluid pressure exceeds the downstream fluid pressure and close when the downstream fluid pressure exceeds the upstream fluid pressure. The free edges of the leaflets coapt under the influence of downstream fluid pressure closing the valve to prevent downstream blood from flowing retrograde through the valve.

Valve durability under the repetitive loads of the leaflets opening and closing is dependent, in part, on the dynamic characteristics of the leaflets. Thin leaflets can develop folds that repeatedly form in the central portion of the leaflet during the opening and closing action of the valve, often times resulting in the formation of a hole within the leaflet at a site of repeated bending stress.

One contribution to the heretofore insurmountable problem of developing a successful synthetic leaflet valve is that synthetic leaf bending appears to be a chaotic process. Each leaflet takes a characteristic bending shape that is repeated with each cycle, but each leaflet's characteristic bending shape is different from an adjacent leaflet. In some cases, the leaflet bending profile is a large-radius, continuous, three-dimensional curve. In others, however, particularly in very thin materials, tight radius bends appear in the form of out-of-plane buckling imposing high strains resulting in leaflet failure.

Therefore, there exists a need for thin leaflet prosthetic valves that exhibit improved longevity while still providing equal, or better yet, improved hemodynamic performance when compared with valves that have heretofore been developed.

### SUMMARY

In accordance with embodiments, the present invention comprises apparatus and systems for valve replacement or augmentation, such as cardiac valve replacement. The present invention is directed towards leaflet design or modifications and leaflet-type cardiac valves that not only improve upon conventional prosthetic valve hemodynamics, but also reduce the incidence of premature leaflet failure. Stated otherwise, the leaflet designs contemplated herein demonstrate improved performance and improved longevity in valve leaflets that would otherwise exhibit tight-radius buckling.

In accordance with other embodiments, a leaflet comprises a guiding element that improves both the longevity and the hemodynamic performance by stabilizing the motion of the leaflet. The guiding element is operable to control the bending patterns or shapes assumed by the leaflet as it moves between open and closed positions. Additionally, the guiding element is operable to minimize or eliminate tight-radius bending, buckling, wrinkling, and other undesirable folding in the central portion of the leaflet, thus contributing to both its hemodynamic performance and its longevity.

In accordance with other embodiments, a leaflet for a prosthetic valve comprises a plurality of layers of film coupled together and configured in the form of the leaflet. One or more guiding elements are coupled between two of the plurality of layers of film, wherein the guiding element is relatively more stiff compared to the plurality of layers of film.

In accordance with other embodiments, a prosthetic valve comprises a frame, at least one leaflet, and a guiding element. Each leaflet comprises a plurality of layers of film coupled together. Each leaflet defines a leaflet base, a leaflet edge portion opposite the leaflet base, and a central portion between the leaflet base and the leaflet edge portion. The leaflet is coupled to the frame along at least a portion of the leaflet base. The guiding element is coupled between two of the plurality of layers of film that the leaflet is made. The guiding element is located in the central portion and spaced apart from the frame. The guiding element is relatively more stiff compared to the plurality of layers of film.

In accordance with other embodiments, a prosthetic valve comprises a frame, at least one leaflet, and a guiding element. Each leaflet comprises a plurality of layers of film coupled together. Each leaflet defines a leaflet base, a leaflet edge portion opposite the leaflet base, and a central portion between the leaflet base and the leaflet edge portion. Each leaflet is coupled to the frame along at least a portion of the leaflet base. Each leaflet is pivotable between an open position and a closed position. The central portion has a greater stiffness than at least one of the leaflet edge portion and the leaflet base.

In accordance with other embodiments, a leaflet for a prosthetic valve comprises a plurality of layers of film coupled together and configured in the form of the leaflet and one or more guiding elements coupled between two of the plurality of layers of film. The leaflet defines a leaflet edge portion and a leaflet base opposite from the leaflet edge portion and a central portion between the leaflet edge portion and the leaflet base. The guiding element is located in the central portion. The guiding element is relatively more stiff compared to the plurality of layers of film. The one or more guiding elements have a length which is aligned radiating away from but spaced apart from the leaflet base such that the leaflet pivots substantially from the leaflet base when the leaflet is deployed in the prosthetic valve and the prosthetic valve is operated so as to flex the leaflet.

### BRIEF DESCRIPTION OF THE DRAWINGS

Embodiments will be described in conjunction with the accompanying drawing figures in which like numerals denote like elements and:

FIG. 1A is a top view of an embodiment of a valve in a closed configuration, in accordance with an embodiment;

FIG. 1B is an axial view of an embodiment of the valve of FIG. 1A in an open configuration, in accordance with an embodiment;

FIG. 2 is a perspective view of an embodiment of a valve in a closed configuration, in accordance with an embodiment;

FIG. 3 is a perspective view of an embodiment of a valve frame, in accordance with an embodiment;

FIG. 4A is a perspective view of an embodiment of a valve in a closed configuration having a leaflet with a guiding element, in accordance with an embodiment;

FIG. 4B is an axial view of the embodiment of the valve of FIG. 4A;

FIG. 4C is an axial view photo of the embodiment of the valve of FIG. 4A;

FIG. 5 is an axial view of an embodiment of a valve having leaflets comprising a guiding element, in accordance with an embodiment;

FIG. 6 is an axial view of an embodiment of a valve having leaflets comprising a guiding element, in accordance with an embodiment;

FIG. 7 is an axial view of an embodiment of a valve having leaflets comprising a guiding element and two guiding elements, in accordance with an embodiment;

FIG. 8 is an axial view of an embodiment of a valve having leaflets comprising five guiding elements, in accordance with an embodiment;

FIG. 9 is an axial view of an embodiment of a valve having leaflets comprising a central guiding element and two side guiding elements, in accordance with an embodiment;

FIG. 10 is an axial view of an embodiment of a valve having leaflets comprising a guiding element;

FIG. 11 is a side perspective view of a leaflet frame coupled to a mandrel in the process of having a film wound thereon defining layers, with a guiding element contained between at least two of the layers of film, in accordance with an embodiment; and

FIG. 12 is a cross-sectional view of a guiding element between layers of film, in accordance with an embodiment.

### DETAILED DESCRIPTION

Persons skilled in the art will readily appreciate that various aspects of the present invention may be realized by any number of methods and apparatus configured to perform the intended functions. Stated differently, other methods and apparatus may be incorporated herein to perform the intended functions. It should also be noted that the accompanying drawing figures referred to herein are not all drawn to scale, but may be exaggerated to illustrate various aspects of the present invention, and in that regard, the drawing figures should not be construed as limiting.

Although the embodiments herein may be described in connection with various principles and beliefs, the described embodiments should not be bound by theory. For example, embodiments are described herein in connection with prosthetic valves, more specifically cardiac prosthetic valves. However, embodiments within the scope of this disclosure can be applied toward any valve or mechanism of similar structure and/or function. Furthermore, embodiments within the scope of this disclosure can be applied in non-cardiac applications.

The term leaflet as used herein in the context of prosthetic valves is a flexible component of a one-way valve wherein the leaflet is operable to move between an open and closed position under the influence of a pressure differential. In the open position the leaflet allows blood to flow through the valve. In the closed position the leaflet substantially blocks retrograde flow through the valve. In embodiments comprising multiple leaflets, each leaflet cooperates with at least one neighboring leaflet to block the retrograde flow of blood. The pressure differential in the blood is caused, for example, by the contraction of a ventricle or atrium of the heart, such pressure differential typically resulting from a fluid pressure building up on one side of the leaflets when closed. As the pressure on the inflow side of the valve rises above the pressure on the outflow side of the valve, the leaflets open and blood flows therethrough. As blood flows through the valve into a neighboring chamber or blood vessel, the pressure on the inflow side equalizes with the pressure on the outflow side. As the pressure on the outflow side of the valve raises above the blood pressure on the inflow side of the valve, the leaflet returns to the closed position generally preventing retrograde flow of blood through the valve.

The term membrane as used herein refers to a sheet of material comprising a single composition, such as, but not limited to, expanded fluoropolymer and synthetic polymer having a structure defining fibers, such as, but not limited to, porous polyethylene.

The term composite material as used herein refers to a combination of a membrane, such as, but not limited to, expanded fluoropolymer, and an elastomer, such as, but not limited to, a fluoroelastomer. The elastomer can be imbibed within a porous structure of the membrane, coated on one or both sides of the membrane, or a combination of coated on and imbibed within the membrane.

The term laminate as used herein refers to multiple layers of membrane, composite material, or other materials, such as elastomer, and combinations thereof.

The term film as used herein generically refers to one or more of the membrane, composite material, or laminate.

The term leaflet window is defined as that space that a frame defines from which a leaflet extends. The leaflet may extend from frame elements or adjacent to frame elements and spaced apart therefrom.

The terms native valve orifice and tissue orifice refer to an anatomical structure into which a prosthetic valve may be placed. Such anatomical structure includes, but is not limited to, a location wherein a cardiac valve may or may not have been surgically removed. It is understood that other anatomical structures that can receive a prosthetic valve include, but are not limited to, veins, arteries, ducts and shunts. It is further understood that a valve orifice or implant site may also refer to a location in a synthetic or biological conduit that may receive a valve.

As used herein, “couple” means to join, connect, attach, adhere, affix, or bond, whether directly or indirectly, and whether permanently or temporarily.

Embodiments herein include various apparatus, systems, and methods for a prosthetic valve suitable for, such as, but not limited to, cardiac valve replacement. The valve is operable as a one-way valve wherein the valve defines a valve orifice into which leaflets open to permit flow and close so as to occlude the valve orifice and prevent retrograde flow.

Embodiments are directed to an apparatus and system for valve replacement or augmentation, such as cardiac valve replacement. The present embodiments are directed towards leaflet design or modifications and leaflet-type cardiac valves that not only improve upon conventional prosthetic valve hemodynamics, but also reduce leaflet fatigue and failure. Stated otherwise, the leaflet embodiments presented herein provide improved leaflet bending, and thereby improved lifetime and improved hemodynamics.

Embodiments provided herein are related to prosthetic heart valve leaflets comprising one or more guiding elements that allow for control of the movement of the leaflets, such as, but not limited to, controlling the bending characteristics of the leaflet.

In accordance with embodiments presented herein, a prosthetic valve comprises a plurality of polymer leaflets. The polymer leaflets comprise a laminate of multiple layers of membrane, composite material, or other materials, such as elastomer, and combinations thereof. One or more guiding elements are coupled to and contained within the laminate lying between two of the multiple layers of membrane or composite material. The guiding element is operable to provide a structural influence on the leaflet such as to control the bending characteristics of the leaflet. Since the guiding element is fully contained within the laminate layers, the guiding element remains permanently coupled to the leaflet. Further, since the guiding element is fully contained within the laminate layers, the guiding element is not exposed to the blood stream.

Another embodiment is directed towards a prosthetic valve comprising a leaflet support member and at least one leaflet as described above wherein the leaflet is connected to the support member along the base portion of the leaflet. The leaflet is movable between a first position and a second position, such that in the first position, the valve is a flow occluder and in the second position, the valve is a flow orifice. The valve further comprises a guiding element, as described herein, connected to at least one leaflet. Similarly, in a valve embodiment comprising multiple leaflets 140, at least one leaflet 140 may not have a guiding element 150, while at least one leaflet does comprise a guiding element 150.

In a further embodiment, the valve comprises a compressed configuration and an expanded configuration. As such, a valve can be compressible or crushable under the application of a binding or compression force to obtain a compressed configuration. However, once the force is removed, the expanded configuration of the valve as it was prior to the compression is substantially retained. To this end, a support member may comprise a shape memory material. A compressible valve may be implanted via endovascular techniques now known or hereinafter derived.

### Valve

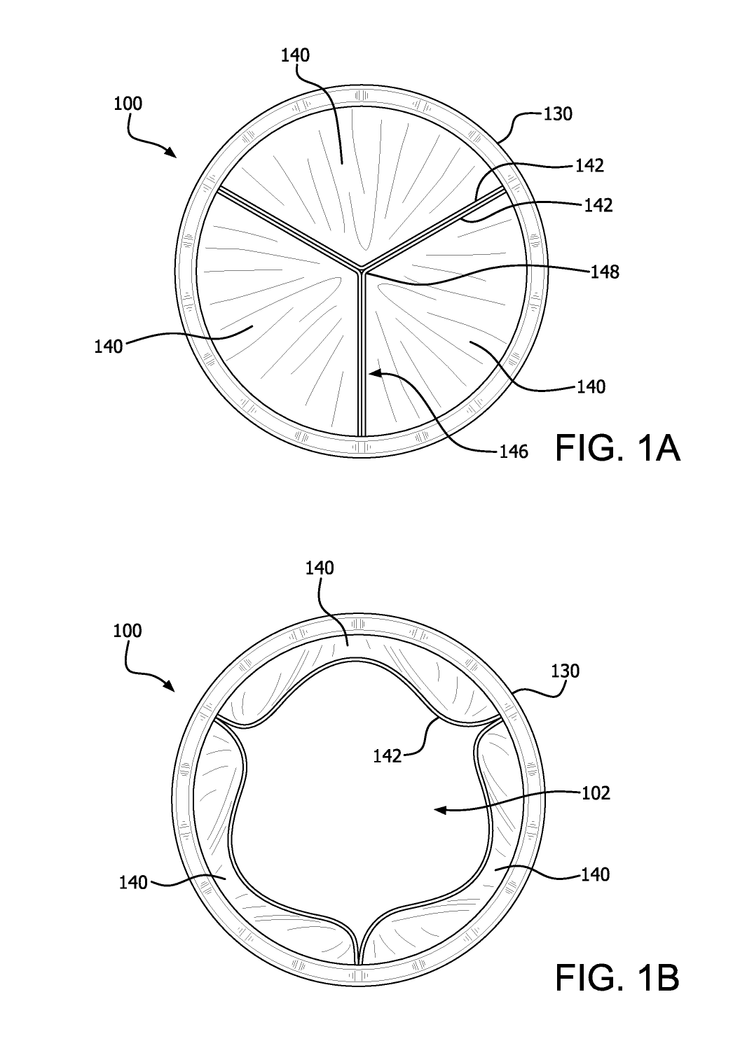

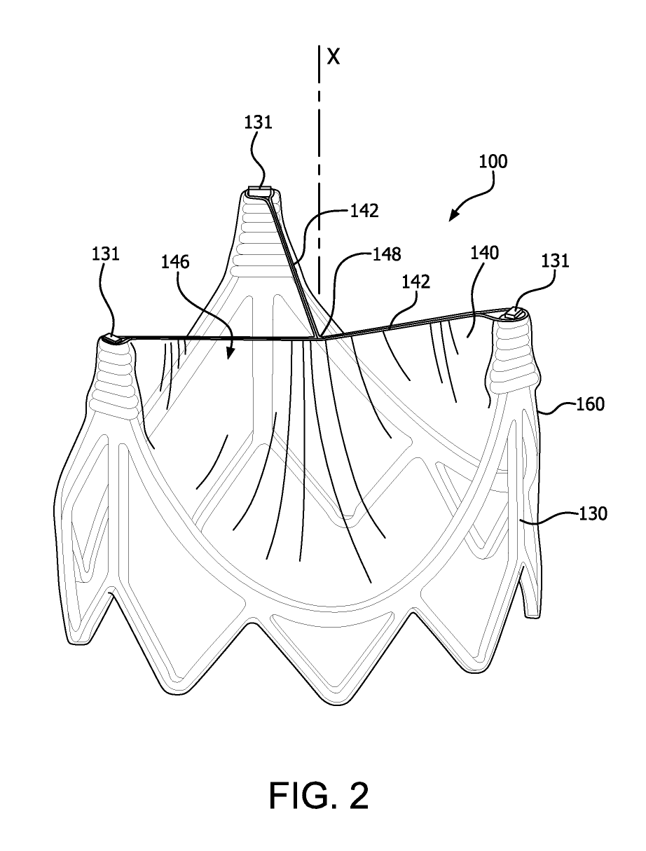

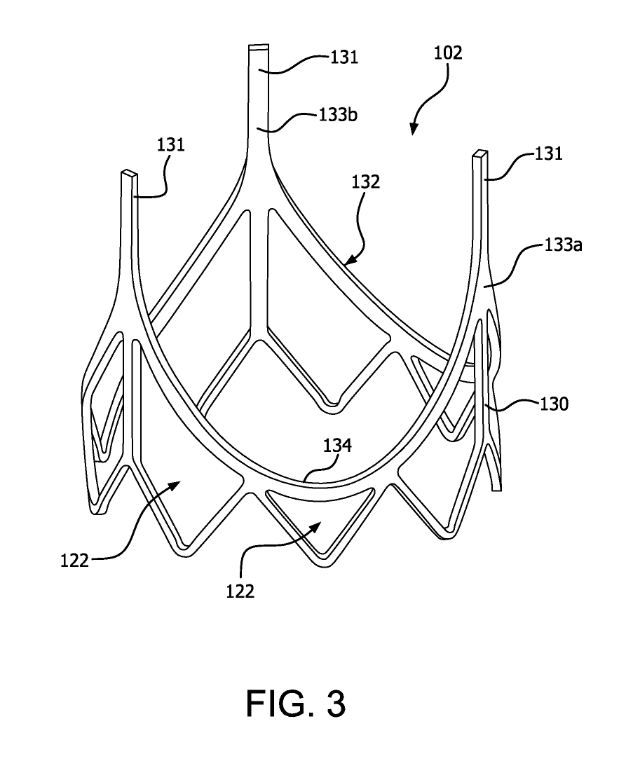

FIGS. 1A and 1B are axial views of a valve 100 in the closed and open condition, respectively, in accordance with an embodiment. FIG. 2 is a perspective view of the valve 100 in the closed condition. The valve 100 comprises a frame 130 and a film 160 covering the frame 130 forming the leaflets 140 coupled to the frame 130, in accordance with an embodiment. FIG. 3 is a perspective view of the frame 130, in accordance with an embodiment.

### Film

The film 160 that makes up the leaflet 140 can comprise any biocompatible material sufficiently compliant and flexible, such as a biocompatible polymer. The film 162 can comprise a membrane that is combined with an elastomer to form a composite material. The film 160, according to an embodiment, includes a composite material comprising an expanded fluoropolymer membrane, which comprises a plurality of spaces within a matrix of fibrils, and an elastomeric material. It should be appreciated that multiple types of fluoropolymer membranes and multiple types of elastomeric materials can be combined to form a laminate while remaining within the scope of the present disclosure. It should also be appreciated that the elastomeric material can include multiple elastomers, multiple types of non-elastomeric components, such as inorganic fillers, therapeutic agents, radiopaque markers, and the like while remaining within the scope of the present disclosure.

A film 160 generically refers to one or more of the membrane, composite material, or laminate as previously defined. The leaflets 140 are comprised of the film 160. Details of various types of film 160 are discussed below. In an embodiment, the film 160 can be formed from a generally tubular material to couple the frame 130 and to form the leaflets 140. As will be discussed below, the laminate comprises a number of layers of membrane and/or composite material, with the guiding element 150 being coupled and contained within at least two layers of membrane and/or composite material.

In an embodiment, the film 160 comprises a biocompatible polymer that is combined with an elastomer, referred to as a composite. A material according to one embodiment includes a composite material comprising an expanded fluoropolymer membrane, which comprises a plurality of spaces within a matrix of fibrils, and an elastomeric material. It should be appreciated that multiple types of fluoropolymer membranes and multiple types of elastomeric materials can be combined to form a laminate while remaining within the scope of the present disclosure. It should also be appreciated that the elastomeric material can include multiple elastomers, multiple types of non-elastomeric components, such as inorganic fillers, therapeutic agents, radiopaque materials, and the like while remaining within the scope of the present disclosure.

In accordance with an embodiment, the composite material includes an expanded fluoropolymer material made from porous ePTFE membrane, for instance as generally described in U.S. Pat. No. 7,306,729 to Bacino.

The expandable fluoropolymer, used to form the expanded fluoropolymer material described, may comprise PTFE homopolymer. In alternative embodiments, blends of PTFE, expandable modified PTFE and/or expanded copolymers of PTFE may be used. Non-limiting examples of suitable fluoropolymer materials are described in, for example, U.S. Pat. No. 5,708,044, to Branca, U.S. Pat. No. 6,541,589, to Baillie, U.S. Pat. No. 7,531,611, to Sabol et al., U.S. patent application Ser. No. 11/906,877, to Ford, and U.S. patent application Ser. No. 12/410,050, to Xu et al.

The expanded fluoropolymer membrane can comprise any suitable microstructure for achieving the desired leaflet performance. In accordance with an embodiment, the expanded fluoropolymer comprises a microstructure of nodes interconnected by fibrils, such as described in U.S. Pat. No. 3,953,566 to Gore. The fibrils radially extend from the nodes in a plurality of directions, and the membrane has a generally homogeneous structure. Membranes having this microstructure may typically exhibit a ratio of matrix tensile strength in two orthogonal directions of less than 2, and possibly less than 1.5.

In another embodiment, the expanded fluoropolymer membrane has a microstructure of substantially only fibrils, as is generally taught by U.S. Pat. No. 7,306,729, to Bacino. The expanded fluoropolymer membrane having substantially only fibrils, can possess a high surface area, such as greater than 20 m²/g, or greater than 25 m²/g, and in some embodiments can provide a highly balanced strength material having a product of matrix tensile strengths in two orthogonal directions of at least 1.5×10⁵ MPa², and/or a ratio of matrix tensile strengths in two orthogonal directions of less than 4, and possibly less than 1.5.

The expanded fluoropolymer membrane can be tailored to have any suitable thickness and mass to achieve the desired leaflet performance. By way of example, but not limited thereto, the leaflet 140 comprises an expanded fluoropolymer membrane having a thickness of about 0.1 μm. The expanded fluoropolymer membrane can possess a mass per area of about 1.15 g/m². Membranes according to an embodiment of the invention can have matrix tensile strengths of about 411 MPa in the longitudinal direction and 315 MPa in the transverse direction.

Additional materials may be incorporated into the pores or within the material of the membranes or in between layers of membranes to enhance desired properties of the leaflet. Composite materials described herein can be tailored to have any suitable thickness and mass to achieve the desired leaflet performance. Composite materials according to embodiments can include fluoropolymer membranes and have a thickness of about 1.9 μm and a mass per area of about 4.1 g/m².

The expanded fluoropolymer membrane combined with elastomer to form a composite material provides the elements of the present disclosure with the performance attributes required for use in high-cycle flexural implant applications, such as heart valve leaflets, in various ways. For example, the addition of the elastomer can improve the fatigue performance of the leaflet by eliminating or reducing the stiffening observed with ePTFE-only materials. In addition, it may reduce the likelihood that the material will undergo permanent set deformation, such as wrinkling or creasing, that could result in compromised performance. In one embodiment, the elastomer occupies substantially all of the pore volume or space within the porous structure of the expanded fluoropolymer membrane. In another embodiment the elastomer is present in substantially all of the pores of the at least one fluoropolymer layer. Having elastomer filling the pore volume or present in substantially all of the pores reduces the space in which foreign materials can be undesirably incorporated into the composite. An example of such foreign material is calcium that may be drawn into the membrane from contact with the blood. If calcium becomes incorporated into the composite material, as used in a heart valve leaflet, for example, mechanical damage can occur during cycling open and closed, thus leading to the formation of holes in the leaflet and degradation in hemodynamics.

In an embodiment, the elastomer that is combined with the ePTFE is a thermoplastic copolymer of tetrafluoroethylene (TFE) and perfluoromethyl vinyl ether (PMVE), such as described in U.S. Pat. No. 7,462,675 to Chang et al. As discussed above, the elastomer is combined with the expanded fluoropolymer membrane such that the elastomer occupies substantially all of the void space or pores within the expanded fluoropolymer membrane to form a composite material. This filling of the pores of the expanded fluoropolymer membrane with elastomer can be performed by a variety of methods. In one embodiment, a method of filling the pores of the expanded fluoropolymer membrane includes the steps of dissolving the elastomer in a solvent suitable to create a solution with a viscosity and surface tension that is appropriate to partially or fully flow into the pores of the expanded fluoropolymer membrane and allow the solvent to evaporate, leaving the filler behind.

In one embodiment, the composite material comprises three layers: two outer layers of ePTFE and an inner layer of a fluoroelastomer disposed therebetween. Additional fluoroelastomers can be suitable and are described in U.S. Publication No. 2004/0024448 to Chang et al.

In another embodiment, a method of filling the pores of the expanded fluoropolymer membrane includes the steps of delivering the filler via a dispersion to partially or fully fill the pores of the expanded fluoropolymer membrane.

In another embodiment, a method of filling the pores of the expanded fluoropolymer membrane includes the steps of bringing the porous expanded fluoropolymer membrane into contact with a sheet of the elastomer under conditions of heat and/or pressure that allow elastomer to flow into the pores of the expanded fluoropolymer membrane.

In another embodiment, a method of filling the pores of the expanded fluoropolymer membrane includes the steps of polymerizing the elastomer within the pores of the expanded fluoropolymer membrane by first filling the pores with a prepolymer of the elastomer and then at least partially curing the elastomer.

After reaching a minimum percent by weight of elastomer, the leaflets constructed from fluoropolymer materials or ePTFE generally performed better with increasing percentages of elastomer resulting in significantly increased cycle lives. In one embodiment, the elastomer combined with the ePTFE is a thermoplastic copolymer of tetrafluoroethylene and perfluoromethyl vinyl ether, such as described in U.S. Pat. No. 7,462,675 to Chang et al., and other references that would be known to those of skill in the art. Other biocompatible polymers which can be suitable for use in leaflet 140 include but are not limited to the groups of urethanes, silicones(organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

### Frame

FIG. 3 is a perspective view of the frame 130 in the embodiment of FIGS. 1A and 1B. The frame 130 is a generally tubular member defining a valve orifice 102 and providing structural, load-bearing support to the leaflet 140. In addition, the frame 130 can be configured to provide positive engagement to the recipient tissue at the implantation site.

The frame 130 can comprise any metallic or polymeric biocompatible material. For example, the frame 130 can comprise a material, such as, but not limited to nitinol, cobalt-nickel alloy, stainless steel, and polypropylene, acetyl homopolymer, acetyl copolymer, ePTFE, other alloys or polymers, or any other biocompatible material having adequate physical and mechanical properties to function as described herein.

By way of example, and as illustrated in the embodiments of FIGS. 1A-B, 2 and 3, the frame 130 defines a stent having apertures 122. The open framework of the stent can define any number of features, repeatable or otherwise, such as geometric shapes and/or linear or meandering series of sinusoids. An open framework can be etched, cut, laser cut, or stamped into a tube or a sheet of material, with the sheet then formed into a substantially cylindrical structure. In other embodiments, the frame 130 can have a solid wall. Alternatively, an elongated material, such as a wire, bendable strip, or a series thereof, can be bent or braided and formed into a substantially cylindrical structure. For example, the frame 130 can comprise a stent or stent graft type structure known in the art.

In accordance with embodiments, the frame 130 can be configured to provide positive engagement to an implant site. In another embodiment, the valve 100 further includes a sewing cuff (not shown) coupled about the frame 130, that is operable to accept suture so as to be sewn to a tissue orifice as is known in the art. It is understood that conventional surgical and transcatheter techniques to implant prosthetic valves can be used to implant the valve 100.

The frame 130 comprises three interconnected U-shaped portions 132. Each of the U-shaped portions 132 defines a base 134. The U-shaped portions 132 intersect with an adjacent U-shaped portion defining a post 131. The frame 130 as shown in FIG. 3 comprised three U-shaped portions 132 and three posts 131, upon each of which a leaflet 140 is coupled as shown in FIG. 2.

The frame 130 can comprise, such as, but not limited to, an elastically deformable metallic or polymeric biocompatible material. The frame 130 can comprise a shape-memory material, such as nitinol, a nickel-titanium alloy. Other materials suitable for the frame 130 include, but not limited to, other titanium alloys, stainless steel, cobalt-nickel alloy, polypropylene, acetyl homopolymer, acetyl copolymer, other alloys or polymers, or any other biocompatible material having adequate physical and mechanical properties to function as a frame 130 as described herein.

### Leaflet

Each of the U-shaped portions 132 of the frame 130 is provided with a biocompatible material, such as the film 162 which can be coupled to the frame outside surface 133a and the frame inside surface 133b of the frame 130; wherein the film 162 defines a leaflet 140. Each leaflet 140 defines a leaflet free edge 142 that is not coupled to the frame 130.

In accordance with an embodiment, the leaflet 140 can comprise a biocompatible material that is not of a biological source and that is sufficiently compliant and strong for the particular purpose, such as a biocompatible polymer. In an embodiment, the leaflet 140 comprises a membrane that is combined with an elastomer to form a composite material.

The shape of the leaflets 140 are defined at least in part by the shape of the frame 130 and the leaflet free edge 142. The shape of the leaflets 140 can also be defined, at least in part, by guiding elements 150 as described below. The shape of the leaflets 140 can also be defined, at least in part, by processes used to manufacture the valve 100, such as, but not limited to a molding and trimming processes to impart a predetermined shape to the leaflet 140.

Fluid flow is permitted through the valve orifice 102 when the leaflets 140 are in an open position as shown in FIG. 2. The leaflets 140 generally flex about the base 134 of the U-shaped portion 132 as the leaflets 140 open and close. In an embodiment, when the valve 100 is closed, generally about half of each leaflet free edge 142 abuts an adjacent half of a leaflet free edge 142 of an adjacent leaflet 140, as shown in FIG. 2. The three leaflets 140 of the embodiment of FIGS. 1A and 2 meet at a triple point 148. The valve orifice 102 is occluded when the leaflets 140 are in the closed position stopping fluid flow.

The leaflet 140 can be configured to actuate at a pressure differential in the blood caused, for example, by the contraction of a ventricle or atrium of the heart, such pressure differential typically resulting from a fluid pressure building up on one side of the valve 100 when closed. As the pressure on an inflow side of the valve 100 rises above the pressure on the outflow side of the valve 100, the leaflet 140 opens and blood flows therethrough. As blood flows through the valve 100 into a neighboring chamber or blood vessel, the pressure equalizes. As the pressure on the outflow side of the valve 100 rises above the blood pressure on the inflow side of the valve 100, the leaflet 140 returns to the closed position generally preventing the retrograde flow of blood through the inflow side of the valve 100.

It is understood that the frame 130 can comprise any number of U-shaped portions 132, and thus leaflets 140, suitable for a particular purpose. Frames 130 comprising one, two, three or more U-shaped portions 132 and corresponding leaflets 140 are appreciated.

It is appreciated that the film 160 can be coupled to the frame 130 in many ways suitable for a particular purpose. By way of example, and not limited thereto, the frame 130 can be wrapped with overlapping layers of the film 160. The film 160 can be coupled to the frame outside surface 133a or the frame inside surface 133b of the frame 130. In another embodiment, the film 160 can be coupled to either of the frame outside surface 133a or the frame inside surface 133b.

The film 160 can be configured to prevent blood from traveling through or across the valve 100 other than through the valve orifice 102 when the leaflets 140 are in an open position. As such, the film 160 creates a barrier to blood flow in any interstitial space(s), such as apertures 122 shown in FIG. 3, of the frame 130 that the film 160 covers.

The film 160 is fixedly secured or otherwise coupled at a single or a plurality of locations of the frame outside surface 133a and the frame inside surface 133b of the frame 130, for example, using one or more of taping, heat shrinking, adhesion and other processes known in the art. In some embodiments, a plurality of membrane/composite layers, such as, but not limited to a laminate, are used and can be coupled to the frame 130 to form at least a portion of the film 160.

### Leaflet Dynamics

A leaflet 140 in accordance with the present embodiments as used in the context of cardiac valves is configured to move between an open and closed position which allows blood to flow when open and which substantially blocks retrograde flow of blood when closed. In embodiments comprising multiple leaflets 140, a leaflet 140 cooperates with at least one neighboring leaflet 140 to block retrograde flow of blood and each leaflet is coupled to a support member, such as, but not limited to, pivotally or rotatably mounted to the frame 130.

Fluid flow is permitted through the valve orifice 102 when the leaflets 140 are in an open position as shown in FIG. 1B. The leaflets 140 generally flex about the base 134 of the U-shaped portion 132 as the leaflets 140 open and close, as shown in FIG. 3. In an embodiment, when the valve 100 is closed, generally about half of each leaflet free edge 142 abuts an adjacent half of a leaflet free edge 142 of an adjacent leaflet 140, as shown in FIG. 2. The three leaflets 140 of the embodiment of FIGS. 1A and 2 meet at a triple point 148. The valve orifice 102 is occluded when the leaflets 140 are in the closed position stopping fluid flow.

The leaflet 140 can be configured to actuate at a pressure differential in the blood caused, for example, by the contraction of a ventricle or atrium of the heart, such pressure differential typically resulting from a fluid pressure building up on one side of the valve 100 when closed. As the pressure on an inflow side of the valve 100 rises above the pressure on the outflow side of the valve 100, the leaflet 140 opens and blood flows therethrough. As blood flows through the valve 100 into a neighboring chamber or blood vessel, the pressure equalizes. As the pressure on the outflow side of the valve 100 rises above the blood pressure on the inflow side of the valve 100, the leaflet 140 returns to the closed position generally preventing the retrograde flow of blood through the inflow side of the valve 100.

For purposes of cardiac valves, a leaflet thickness may range from about 10 μm to about 100 μm but again such thickness may vary from the above stated ranges depending on the size, material, and desired function of the leaflet. As discussed below, improvements in accordance with the present embodiments may provide for leaflet thicknesses outside of conventional thicknesses.

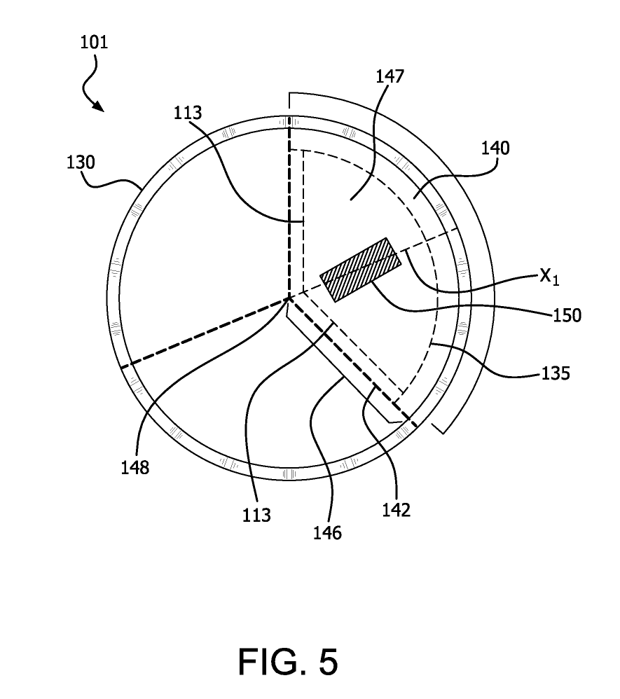

FIG. 5 is an axial view of a representation of a valve 101. A leaflet edge portion 113 comprises a coaptation region 146 of the leaflet 140. A central portion 147 comprises an area between a leaflet base 135 and the leaflet edge portion 113. A coaptation region 146 is the area comprising the junction formed between two leaflets 140 in the closed position. The leaflet 140 also comprises a vertical axis X1. The height of the leaflet 140 is the length of the leaflet 140 along a line parallel to the vertical axis X1. The width of the leaflet 140 is the length of the leaflet 140 along a line perpendicular to the vertical axis X1, which may vary between the leaflet base 135 and the leaflet free edge 142. The guiding element defines a guiding element length and the leaflet defines a leaflet length extending from the leaflet base and the edge portion, the guiding element length being less than the leaflet length.

### Guiding Elements

Embodiments of leaflets presented herein comprise one or more guiding elements that are operable to control the movement of the leaflet in a predetermined way.

The guiding element improves both the longevity, such as, but not limited to, durability, and the hemodynamic performance of the valve.

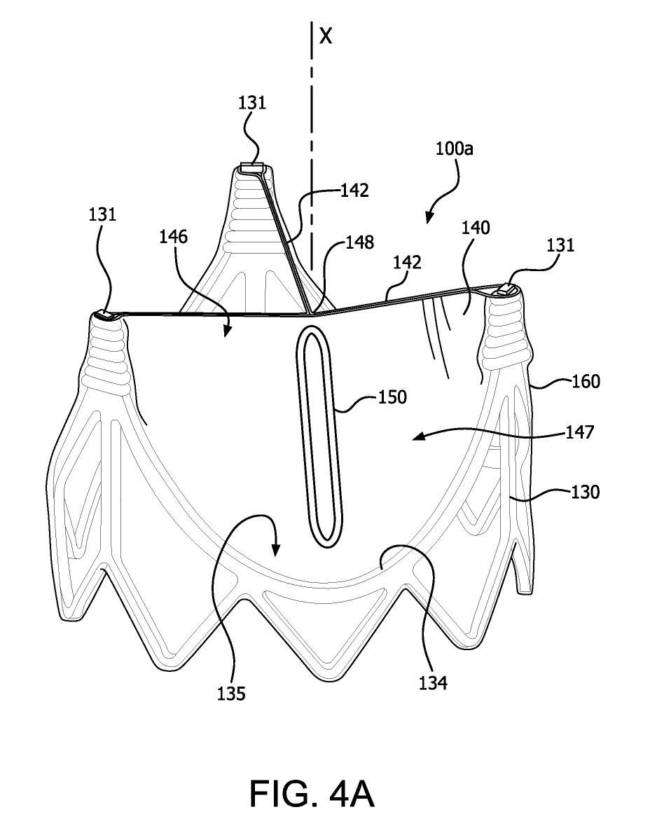

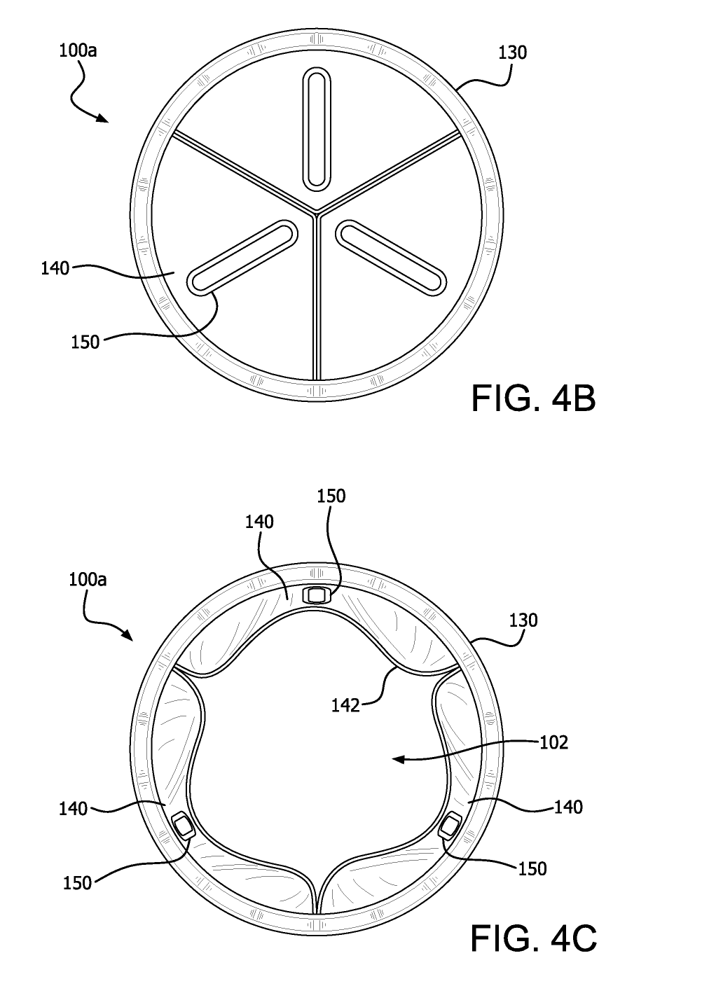

In accordance with an embodiment, the leaflet further comprises a guiding element 150 as shown in FIGS. 4A-4D and 5. A guiding element 150 is an element within the leaflet 140 that stabilizes the motion of the leaflet 140 and/or affects the bending patterns or shapes assumed by the leaflet 140 as it moves between the open and closed positions as shown in FIGS. 4B and 4C. Similarly, the guiding element 150 may be a load distribution element operable for distributing the load more evenly through the central portion 147 of the leaflet 140.

In accordance with embodiments, the guiding element 150 is an element disposed in the central portion 147 of the leaflet 140, spaced apart from the leaflet base 135 and spaced apart from the frame 130, that is operable to resistant deformation, such as bending along or proximate to the vertical axis X1 that contains the guiding element 150, and as such, shifts a majority of the bending from the central portion 147 towards the leaflet edge portion 113 and to the leaflet base 135 of the leaflet 140. By resisting such deformation, the central portion 147 pivots in a substantially more predictable manner between a substantially closed to open position, or vice versa. For example, the guiding elements 150 in accordance with the present embodiments may facilitate pivoting of the central portion 147 relative to the leaflet base 135 in a substantially planar manner, as opposed to “rolling” open. By so doing, issues such as tight radius bending, buckling, undesirable folding or wrinkling, and the like, as well as other durability and longevity-decreasing occurrences, are minimized or eliminated. In various embodiments, the motion of the central portion 147 of the leaflet 140 between the first position and the second position substantially follows the guiding element 150.

Referring to FIG. 5, the guiding element 150 is located on or within the central portion 147 of the leaflet 140, spaced apart from the leaflet base 135 and spaced apart from the frame 130. The guiding element 150 is operable to stabilize, minimize, or prevent leaflet deformation during leaflet movement between the open position and the closed position as shown in FIGS. 4B and 4C. In an embodiment, a majority of the guiding element 150 is locatable on or within the central portion 147 and crosses, or is coincident with, the vertical axis X1. In an embodiment, the guiding element 150 has a height less than the leaflet height and a width through a point along the vertical axis X1 less than the leaflet's width through that same point. Stated differently, the guiding element 150 in embodiments does not extend all the way to a particular edge of the leaflet 140, such as, but not limited to the leaflet base 135. In embodiments, the leaflet edge portion 113 and/or the leaflet base 135 are free from any portion of the guiding element 150. In an embodiment, the leaflet 140 may comprise the guiding element 150 substantially coincident with the vertical axis X1 and ranging up to the line of coaptation on the axis down to at least half the distance to the leaflet base 135. In various embodiments, the guiding element 150 may have a vertical dimension longer or shorter than the dimension in the orthogonal direction.

With the addition of the guiding element 150, the amplitude or number of sigmoid, or S-shaped, curves formed on the leaflet about the vertical axis X1 during the transition may be reduced, and a majority of such curves are formed closer to the leaflet edge portion 113 and the leaflet base 135 of the leaflet 140 and in a more controlled manner.

For example, the motion of a point on the guiding element 150, as the leaflet 140 moves from a first position to a second position, may exist substantially on a plane, substantially on an arc, and in an embodiment, substantially on an elliptical arc. The motion of the guiding element 150 as a whole tracks a substantially planar pivoting surface while transitioning between the first position and the second position.

In an embodiment, a guiding element 150 may comprise any shape, any configuration, or any material configured to resist leaflet deformation described above about the vertical axis, and in accordance with an embodiment, over a majority of the central portion. For example, with reference to FIGS. 4B, 6-10, a guiding element 150 may comprise at least one of a wire, or otherwise comprises an area of greater stiffness than areas without a guiding element 150.

It is believed that despite the presence of additional mass added to the leaflet 140, the leaflet 140 comprising the guiding element 150 is more responsive to changes in fluid pressure because bending occurs primarily at the leaflet edge portion 113 and the leaflet base 135 of the leaflet 140 rather than occurring first through undesirable bending and buckling in the central portion of the leaflet, as would be the case without a guiding element. Furthermore, as mentioned above, by minimizing planar buckling, the likelihood of leaflet failure is reduced.

In accordance with embodiments, the stiffness of the central portion 147 is increased relative to the leaflet base 135 and leaflet edge portion 113 by at least one of an extra layer of film, a fiber, and a filament located between two of the plurality of layers of film 160 that comprise the leaflet 140, as shown in FIG. 12.

In an embodiment, the shape of the guiding element 150 comprises a wire formed into an oval, such as, but not limited to, a parallel-sided oval, as shown in 4A as guiding element 150. Alternative configurations are appreciates, such as, but not limited to, a polygon, an undulating shape, an S-shape, straight wires, and a figure-eight shape also known as a lemniscate.

FIG. 6 is an axial view of an embodiment of a valve 100b having leaflets 140 comprising a second guiding element 150b. The guiding element 150b has a substantially V shape that is spaced apart from the frame 130 and spans a significant portion of the leaflet 140.

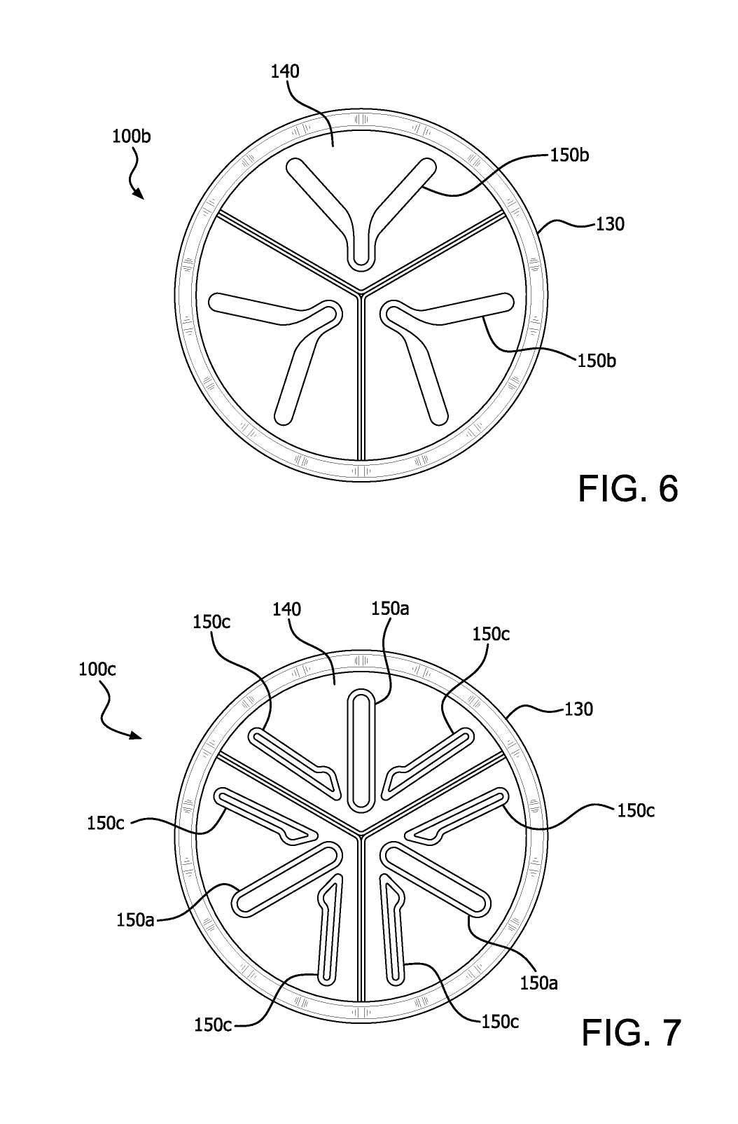

Similarly, a leaflet need not be limited to one guiding element per leaflet. FIG. 7 is an axial view of an embodiment of a second valve 100c having leaflets 140 comprising a first guiding element 150a flanked by a third guiding element 150c on each side of first guiding element 150a. The first guiding element 150a and the third guiding element each have a substantially oval shape and are positioned relative to each other in the leaflet 140 spaced apart from the frame and so as to span a significant portion of the leaflet 140.

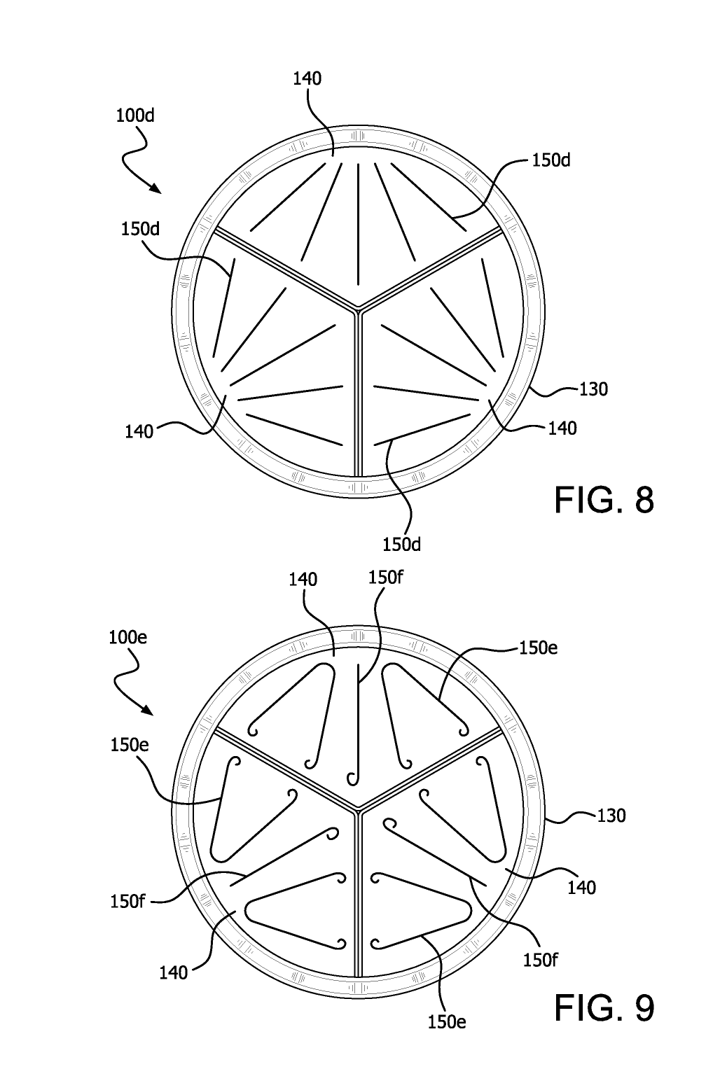

FIG. 8 is an axial view of an embodiment of a valve 100d having leaflets 140 comprising a plurality of fourth guiding elements 150d. Each of the fourth guiding elements 150d is essentially a straight wire or a small diameter rod. The plurality of fourth guiding elements 150d is positioned relative to each other in the leaflet 140 spaced apart from the frame and so as to span a significant portion of the leaflet 140. The fourth guiding elements 150d extend from adjacent the leaflet base toward the leaflet free edge in a fan-like pattern.

FIG. 9 is an axial view of an embodiment of a valve 100e having leaflets 140 comprising a sixth guiding element 150f flanked by a fifth guiding element 150e on each side of sixth guiding element 150f. The sixth guiding element 150f is essentially a straight wire or a small diameter rod having one end bent into a rounded shape. The fifth guiding element 150e is essentially a wire or a small diameter rod bent into a V or U shape having each end bent into a rounded shape. The rounded shape of the ends may help to prevent the ends from penetrating the leaflet causing failure as compared with a sharp point of an un-bent end. The plurality of sixth guiding elements 150f and the fifth guiding element 150e are positioned relative to each other in the leaflet 140 so as to span a significant portion of the leaflet 140. The sixth guiding element 150f and the fifth guiding elements 150e are spaced apart from the frame 130 and extend from adjacent the leaflet base 135 in FIG. 5 toward the leaflet free edge 142 in a fan-like pattern.

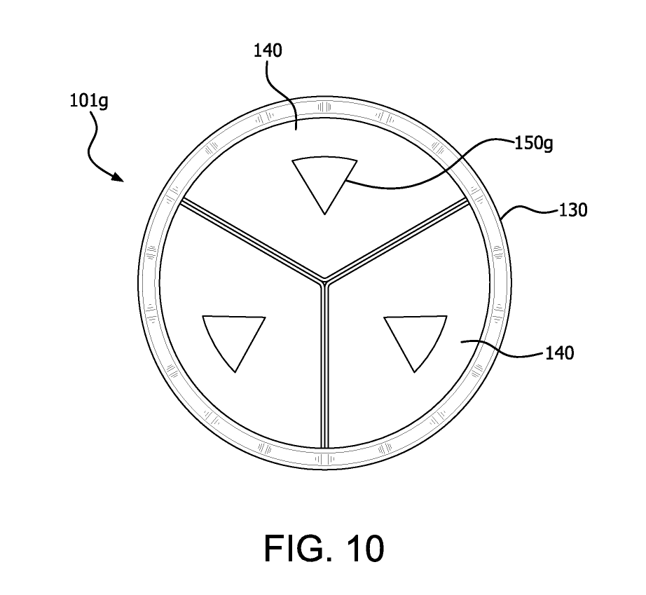

FIG. 10 is an axial view of an embodiment of a valve 100g having leaflets 140 comprising a seventh guiding element 150g. The seventh guiding element 150g has a substantially triangular shape that spans a portion of the leaflet 140 with one side of the triangular shape spaced apart from the frame 130 and adjacent to the leaflet base 135 as shown in FIG. 5.

Any number of guiding elements may be present, and the present embodiments contemplate any guiding element comprising any leaflet modification of any shape or configuration with any material of any combination that stabilizes leaflet motion or resists leaflet deformation on or about the vertical axis, and more over a majority of the central portion.

In accordance with embodiments, the one or more guiding elements 150 have a length which is aligned substantially perpendicular to predetermined stress lines corresponding to lines of stress in the leaflet when the leaflet is deployed in the valve and the valve is operated so as to flex the leaflet. Lines of stress in the leaflet 140 are substantially perpendicular to the lines representing the fourth guiding elements 150d shown in FIG. 8.

The guiding element 150 may comprise any material, including a biocompatible material. For example, the guiding element 150 may comprise a metallic, polymeric, or ceramic material. The guiding element 150 may be of the same or different material from that of the leaflet 140. Such material may comprise a shape memory material such as nitinol. Other materials contemplated include PTFE, such as ePTFE or other fluoropolymers or elastomers, polyurethanes, stainless steel, and other biocompatible materials. In accordance with the present embodiments, the guiding elements 150 may be connected to the surface of the leaflet, embedded therein, such as between layers of leaflet material, or a constituent part thereof.

In an embodiment, the guiding element 150 may comprise a plurality of materials, and thereby exhibit a variable resistance to deformation along its length or width.

In accordance with embodiments, the guiding element defines a shape of one of a polygon, a square-sided oval, an undulating shape, a lemniscate, and an S-type shape.

### Example 1

Referring again to FIGS. 4A-4C, the guiding element 150, in embodiments, adds mass to the leaflet 140. As such, the expected effect would be a slower movement of the leaflet 140 than a leaflet 140 without a guiding element 150. Surprisingly, in embodiments, the leaflet 140 comprising the guiding element 150 has better hemodynamics than the substantially same leaflet without a guiding element. For example, improvements in various performance parameters used to measure hemodynamics from 1.5 fold to 3.3 fold have been observed. As noted previously, such performance parameters may include closing volume, regurgitation fraction (%), elapsed time to open and close, and the amount of pressure drop across the open valve during the positive portion of forward flow. Lower values are indicative of better performance. By adding guiding element 150, the closing volume and regurgitant fraction may be decreased by at least two fold, and similarly, the change in pressure may be decreased nearly two fold. Table 1 below provides an example of actual improved hemodynamics observed by adding the guiding element 150.

TABLE 1

| | Leaflet | Closing | | |
|---|---|---|---|---|
| Guiding | Thickness | Volume | Regurgitant | ΔP |
| Element | (μ) | (ml) | Fraction (%) | (mm/Hg) |
| No | 25 | 9.45 | 11.9 | 7.6 |
| Yes | 25 | 4.46 | 3.6 | 3.9 |

Improved hemodynamics was visually confirmed in that the valve orifice area in the open position was greater with the guiding element 150 than without a guiding element. It was visually confirmed that a valve with guiding element 150 in its open position had a substantially more circular shape. More particularly, the shape formed along the perimeter of the orifice of a cardiac valve in the open position is substantially more circular with the addition of the guiding element 150 than the same valve without a guiding element. It was also observed that the leaflets 140 with the guiding element 150 open and close with less wrinkling and in a more planar fashion in the central portion of the leaflets compared to leaflets 140 without guiding elements.

### Example 2

A valve 100a of FIG. 4A having polymeric leaflets 140 was formed from a film in a form of a composite material having an expanded fluoropolymer membrane and an elastomeric material and joined to a semi-rigid, non-collapsible frame 130, and was constructed according to the following process:

A valve frame was laser machined from a length of MP35N cobalt chromium tube hard tempered with an outside diameter of 26.0 mm and a wall thickness of 0.6 mm in the shape shown in FIG. 3. The frame 130 was electro-polished resulting in 0.0127 mm material removal from each surface and leaving the edges rounded. The frame 130 was exposed to a surface roughening step to improve adherence of leaflets to the frame 130, without degrading fatigue durability performance. The frame was cleaned by submersion in an ultrasonic bath of acetone for approximately five minutes. Plasma treatment of the entire frame surface was performed as commonly known in the arts for cleaning. This treatment also served to improve the wetting of the fluorinated ethylene propylene (FEP) adhesive.

FEP powder (Daikin America, Orangeburg N.Y.) was applied to the frame 130 by first stirring the powder into an airborne “cloud” in a standard kitchen type blender and suspending the frame in the cloud until a uniform layer of powder adhered to the entire surface of the frame 130. The frame 130 was then subjected to a thermal treatment by placing it in a forced air oven set to 320° C. for approximately three minutes. This caused the powder to melt and adhere as a thin coating over the entire frame 130. The frame 130 was removed from the oven and left to cool to room temperature.

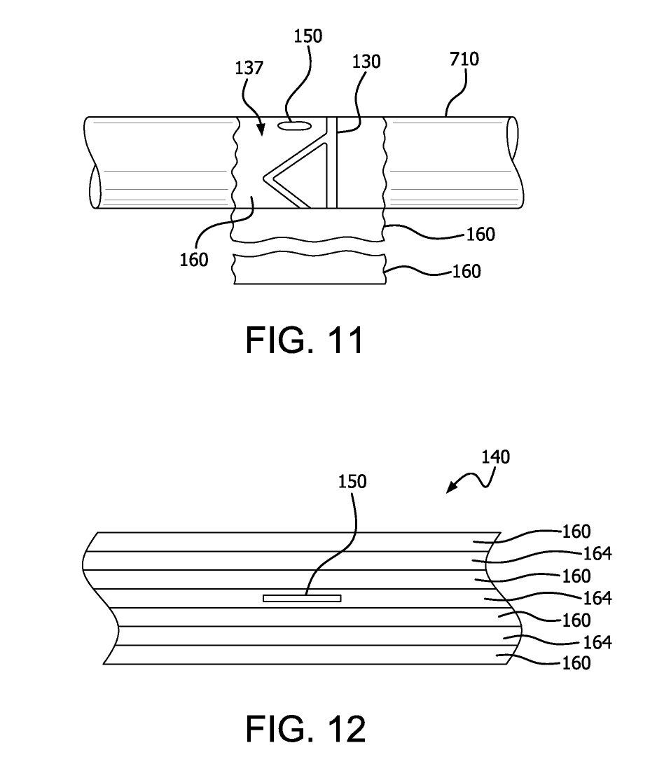

A strain relief and sewing ring (not shown) were attached to the frame 130 in the following manner: a 23 mm diameter cylindrical mandrel was wrapped with a single layer of Kapton® (DuPont) polyimide film and held in place by an adhesive strip of Kapton® tape over the length of the overlapping seam. One wrap of a two layer laminate consisting of an ePTFE membrane laminated to a 25.4 μm thick layer of fluoroelastomer as described below and shown in FIG. 11, was wrapped with the high strength direction along the axis of the Kapton®-covered mandrel 710 with no overlap at the seam. The frame 130 was aligned coaxially over the wrapped mandrel 710. An additional 1 wrap of the two layer laminate was wrapped onto the mandrel encapsulating the entire frame 130 with the seam oriented 180° from the seam of the single inner wrap. The four layer laminate was end cut 135 mm from the base of the frame 130 encapsulated within. The four layer laminate was hand rolled axially in the direction of the base of the frame until the 135 mm length of material constituted approximately a 3 mm outer diameter ring adjacent to the base of the frame. The four layer laminate was end cut approximately 20 mm from the top of the frame and the assembly was compression wrapped helically with two sacrificial layers of ePTFE membrane imbibed with a polyimide, four layers of unsintered ePTFE membrane, and approximately one hundred wraps of an ePTFE fiber. The entire assembly was subjected to a thermal treatment by placing it in a forced air oven set to 280° C. for five minutes and returned to room temperature by immediate water quench upon removal from the oven. The sacrificial layers were removed and the four layer laminate at the top end of the frame trimmed to allow a 2 mm length to extend beyond the perimeter of the top of the frame. The mandrel and Kapton were then removed from the interior of the frame forming a strain relief and sewing ring with the frame laminated within.

A single female mold (not shown) defining the shape of the tri-leaflet was made. Three identical male molds that match the shape and contour of the female mold are held together with a mechanism that enables radial pivoting of the male molds with respect to each other at their base while maintaining both axial and rotational spacing. The female and male molds are wrapped with a single layer of un-sintered ePTFE membrane to act as a cushioning layer and then a single layer of substantially nonporous ePTFE membrane with FEP on one side is used to adhere the membranes together and onto the mandrels with a soldering iron. The sacrificial layers ensure that all the mating surfaces between the male and female molds have a cushioning layer when compressed together; an additional function is as a release layer to prevent the leaflet material from adhering to the molds. The male and female molds are initially combined to create a single cylindrical structure to facilitate leaflet construction and attachment to the frame with strain relief and sewing ring component via a tape wrapping process.

A leaflet material was then prepared. A membrane of ePTFE was manufactured according to the general teachings described in U.S. Pat. No. 7,306,729. The ePTFE membrane had a mass per area of 1.0 g/m2 a matrix tensile strength of 447 MPa in the longitudinal direction and 421 MPa in the transverse direction.

The above membrane was imbibed with a copolymer fluoroelastomer. The copolymer consists essentially of between about 65 and 70 weight percent perfluoromethyl vinyl ether and complementally about 35 and 30 weight percent tetrafluoroethylene. Additional fluoroelastomers may be suitable and are described in U.S. Publication No. 2004/0024448. The fluoroelastomer was dissolved in Novec HFE7500 (3M, St Paul, Minn.) in a 2.5% concentration. The solution was coated using a mayer bar onto the ePTFE membrane (while being supported by a polypropylene release film) and dried in a convection oven set to 145° C. for 30 seconds. After 2 coating steps, the final ePTFE/fluoroelastomer or composite had a mass per area of 6.92 g/m2, 14.4% fluoropolymer by weight, and thickness of 3.22 μm.

Five layers of the composite material were wrapped around the combined molds with the membrane oriented such that the matrix tensile strength of 447 MPa is oriented axially and the elastomer rich side of the composite facing away from the molds.

The subassembly containing the frame 130 with strain relief and sewing ring was aligned both axially and rotationally to match the features of the female mold over the three inner wraps. Ten additional layers of the composite material were wrapped around the combined molds with the membrane oriented such that the matrix tensile strength of 410.9 MPa was oriented axially and the elastomer rich side of the composite facing toward the molds.

FIG. 11 is a simplification of the above method showing a mandrel 710 over which a frame 130 is positioned. The film 160, in the form of composite, is wrapped around the mandrel 710 over the frame 130 forming multiple layers of film 160 with the guiding element 150 of FIG. 4A contained between two of the multiple layers of film 160, as shown in FIG. 12, with area 137 eventually being formed into a leaflet. The leaflet 140 comprises multiple layers of film 160 coupled together with elastomeric material 164 therebetween. FIG. 12 is a cross-section of the leaflet 140 showing the layers of film 160 bound together with elastomeric material 164 therebetween, and the guiding element 150 between two of the multiple layers of film 160.

The male molds were then slid out from underneath the 15-layer composite laminate tube. Each of the male molds was expanded with respect to each other about the pivot at their base. The male mold assembly was coaxially aligned to the female mold facilitating the male molds to compress the cantilevered 15-layer composite laminate tube onto the female tri-leaflet mold surface. Both radial and axial compression were applied by placing a hose clamp over the male molds while simultaneously applying axial load with the translational end of the lathe apparatus.

The assembly consisting of male and female molds, composite laminate, strain relief, frame, and sewing ring was compression wrapped helically with two sacrificial layers of compliant ePTFE membrane imbibed with a polyimide, four layers of un-sintered ePTFE membrane, and approximately one hundred wraps of an ePTFE fiber. The entire assembly was removed from the lathe and placed in a c-clamp fixture to maintain axial compression while subjected to a thermal treatment by placing it in a forced air oven set to 280° C. for 30 minutes. The assembly was removed from the oven and brought back to room temperature via immediate water quench. The sacrificial layers, male, and female molds were removed leaving a fully adhered valve in a closed three dimensional form.

The excess leaflet material was trimmed with scissors from the top of the frame posts to the common triple point of each leaflet to create three commissures or coapting surface regions as depicted in FIG. 4A. The leaflets were opened with an ePTFE mandrel tapered from 10 mm to 25 mm. The round sewing ring at the base of the frame was molded into a flange by placing the valve assembly into a fixture depicted in FIGS. 28a and 28b and using an Branson ultrasonic compression welder (#8400, Branson ultrasonics, Danbury Conn.) with a weld time of 0.8 seconds, hold time of 3.0 seconds, and pneumatic pressure of 0.35 MPa. The ultrasonic welding process was performed twice to create a sewing ring flange thickness of approximately 2 mm with an outer diameter of 33 mm.

The final leaflet was comprised of 14.4% fluoropolymer by weight with a thickness of 58 μm. Each leaflet had 15 layers of the composite and a ratio of thickness/number of layers of 3.87 μm.

The resulting valve assembly includes leaflets formed from a composite material with more than one fluoropolymer layer having a plurality of pores and an elastomer present in substantially all of the pores of the more than one fluoropolymer layer. Each leaflet is capable of being cycled between a closed position, shown illustratively in FIG. 4B, in which blood is prevented from flowing through the valve assembly, and an open position, shown illustratively in FIG. 4C, in which blood is allowed to flow through the valve assembly. Thus, the leaflets of the valve assembly cycle between the closed and open positions generally to regulate blood flow direction in a human patient.

The performance of the valve leaflets in each valve assembly was characterized on a real-time pulse duplicator that measured typical anatomical pressures and flows across the valve The flow performance was characterized by the following process:

1) The valve assembly was potted into a silicone annular ring (support structure) to allow the valve assembly to be subsequently evaluated in a real-time pulse duplicator. The potting process was performed according to the recommendations of the pulse duplicator manufacturer (Vi Vitro Laboratories Inc., Victoria BC, Canada)

2) The potted valve assembly was then placed into a real-time left heart flow pulse duplicator system. The flow pulse duplicator system included the following components supplied by VSI Vivitro Systems Inc., Victoria BC, Canada: a Super Pump, Servo Power Amplifier Part Number SPA 3891; a Super Pump Head, Part Number SPH 5891 B, 38.320 cm2 cylinder area; a valve station/fixture; a Wave Form Generator, TriPack Part Number TP 2001; a Sensor Interface, Part Number VB 2004; a Sensor Amplifier Component, Part Number AM 9991; and a Square Wave Electro Magnetic Flow Meter, Carolina Medical Electronics Inc., East Bend, N.C., USA.

In general, the flow pulse duplicator system uses a fixed displacement, piston pump to produce a desired fluid flow through the valve under test.

3) The heart flow pulse duplicator system was adjusted to produce the desired flow, mean pressure, and simulated pulse rate. The valve under test was then cycled for about 5 to 20 minutes.

4) Pressure and flow data were measured and collected during the test period, including ventricular pressures, aortic pressures, flow rates, and pump piston position.

5) Parameters used to characterize the valve and to compare to post-fatigue values are pressure drop across the open valve during the positive pressure portion of forward flow, effective orifice area, and regurgitant fraction. The values recorded for this valve are displayed in Table x below. All data contained in this table were recorded at 5 liters/min cardiac output at 37 degrees centigrade.

### Example 3

A second valve 100c was constructed as above, except that a first guiding element 150a was flanked by a third guiding element 150c on each side of first guiding element 150a, as shown in FIG. 7, were incorporated into the laminated leaflet construction so that they were contained entirely within each of the three leaflets 140. The first guiding element 150a and the third guiding elements 150c were constructed of 0.151 mm Nitinol wire into elliptical elements. The first guiding element 150a and the third guiding elements 150c were arranged into a pattern radiating from but spaced from the leaflet base 135 of the leaflet 140, shown in FIGS. 5 and 7, and were not attached to the frame 130. The first guiding element 150a was 11.66 mm in length and each third guiding element 150c were 10 mm in length. The first guiding element 150a and the third guiding elements 150c were formed on a pin jig and placed into an oven at 450 degrees centigrade for 10 minutes, removed and water quenched. As above, the valve was loaded into a real-time heart valve tester and performance characteristics measured (see Table 2).

### Example 4

A third valve was constructed as above in example 3, also with 3 0.151 mm guiding elements 150a, 150c, see FIG. 7, made of Nitinol. The central guiding element 150a was configured the same as the central guiding element 150a of example 3 and was 11.43 mm in length. Each of the two side guiding elements or third guiding elements 150c was 8.26 mm in length. None of the 3 guiding elements 150a, 150c were attached directly to the frame 130 and were spaced from the frame 130. These guiding elements 150a, 150c were formed as described in example 3. As above, the valve was loaded into a real-time heart valve tester and performance characteristics measured (see Table 2).

TABLE 2

| | | | | | Closing |
|---|---|---|---|---|---|
| | EOA | Regurgitation | ΔP | Leakage | volume |
| Valve | (cm²) | (%) | (mmHg) | volume (ml) | (ml) |
| Example 3 | 1.9 | 8.7 | 8.8 | 0.4 | 6.3 |
| Example 4 | 1.9 | 5.6 | 8.1 | 2.8 | 1.4 |
| Example 5 | 1.9 | 3.7 | 8.1 | 0.1 | 2.7 |

### Example 5

Another valve identical to that of example 1 was constructed and tested.

### Example 6

This example illustrates the application of non-metallic guiding elements. An additional composite membrane was formed from a composite material comprising a membrane of ePTFE imbibed with a fluoroelastomer, as shown in FIG. 12, leaflet 140. A piece of the film 160 in the form of a composite material approximately 10 cm wide was wrapped onto a circular mandrel to form a tube. The composite material was comprised of three layers: two outer layers of ePTFE and an inner layer of a fluoroelastomer disposed therebetween. The ePTFE membrane was manufactured according to the general teachings described in U.S. Pat. No. 7,306,729. The fluoroelastomer was as in example 2.

The ePTFE membrane had the following properties: thickness=about 15 μm; MTS in the highest strength direction=about 400 MPa; MTS strength in the orthogonal direction=about 250 MPa; Density=about 0.34 g/cm3; IBP=about 660 KPa.

The percent weight of the fluoroelastomer relative to the ePTFE was about 53%.

The multi-layered composite had the following properties: thickness of about 40 μm; density of about 1.2 g/cm3; force to break/width in the highest strength direction=about 0.953 kg/cm; tensile strength in the highest strength direction=about 23.5 MPa (3,400 psi); force to break/width in the orthogonal direction=about 0.87 kg/cm; tensile strength in the orthogonal direction=about 21.4 MPa (3100 psi), and mass/area=about 14 g/m2.

Ten layers of the above composite were heated and compressed together so as to bond to form a single composite. Side elements in the form of dart shapes (not shown) were cut from the 10 layer sheet and were subsequently bonded into the leaflet as in examples 3 and 4. Test results are illustrated below in table 3. Reductions in regurgitation, leakage volume, and closing volume were observed, along with a modestly elevated degree of pressure drop.

TABLE 3

| | | | | | Closing |
|---|---|---|---|---|---|
| | EOA | Regurgitation | ΔP | Leakage | volume |
| Valve | (cm²) | (%) | (mmHg) | volume (ml) | (ml) |
| Example 4 | 2.0 | 11.5 | 7.9 | 3.4 | 5.6 |
| Example 5 | 1.9 | 7.3 | 8.3 | 0.2 | 5.3 |

### Example 7

The purpose of this example is to illustrate that the guiding elements in an embodiment can be employed in valves to be delivered via catheter. Another valve was constructed as in example 3, except that the valve frame employed was of a type that can be diametrically crushed to a small diameter (6 mm), and then, using a balloon, re-expanded to the original diameter of 26 mm. In this case, the material employed to form the leaflet had a weight/area of 0.3 gm/meter², and each layer was 30% ePTFE and 70% PMVE/PTFE copolymer. Fifty layers were used to form the leaflets for a final thickness of about 50 micrometers. The guiding elements were formed and laminated into the leaflets as in example 3.

The results demonstrate that the valve had hemodynamics after crushing/re-expansion very similar to that of before crushing (within measurement error), as shown in Table 4.

TABLE 4

| | | | | Leakage | Closing |
|---|---|---|---|---|---|
| | EOA | Regurgitation | ΔP | volume | volume |
| Valve #7 | (cm²) | (%) | (mmHg) | (ml) | (ml) |
| Before crushing | 2.2 | 10.1 | 6.3 | 4.7 | 3.1 |
| After re- | 2.2 | 10.6 | 7.2 | 4.7 | 3.7 |
| expansion | | | | | |

The foregoing disclosure is merely illustrative of the present invention and is not intended to be construed as limiting the invention. Although one or more embodiments of the present invention have been described, persons skilled in the art will readily appreciate that numerous modifications could be made without departing from the spirit and scope of the present invention. As such, it should be understood that all such modifications are intended to be included within the scope of the present invention.

What is claimed is:

1. A leaflet for a prosthetic valve, comprising:
a plurality of layers of film coupled together and configured in a form of the leaflet; and
one or more guiding elements coupled between two of the plurality of layers of film, wherein each guiding element is relatively more stiff compared to the plurality of layers of film.

2. The leaflet of claim 1, wherein the one or more guiding elements have a length which is aligned substantially perpendicular to predetermined stress lines corresponding to lines of stress in the leaflet when the leaflet is deployed in the valve and the valve is operated so as to flex the leaflet.

3. The leaflet of claim 1 wherein the one or more guiding elements are spaced a predetermined distance from a frame.

4. The leaflet of claim 1, wherein the leaflet defines a leaflet edge portion and a leaflet base opposite from the leaflet edge portion, and a central portion between the leaflet edge portion and the leaflet base, the guiding element being located in the central portion.

5. The prosthetic valve of claim 4, wherein the guiding element defines a guiding element length and the leaflet defines a leaflet length extending from the leaflet base and the leaflet edge portion, the guiding element length being less than the leaflet length.

6. The prosthetic valve of claim 4, the leaflet further comprising a vertical axis and wherein the guiding element crosses the vertical axis.

7. The prosthetic valve of claim 4, the leaflet further comprising a vertical axis and wherein the guiding element is located substantially coincident with at least a portion of the vertical axis.

8. The prosthetic valve of claim 1, wherein the guiding element is comprised of a shape-memory material.

9. The prosthetic valve of claim 1, wherein the guiding element is comprised of a metallic material.

10. The prosthetic valve of claim 8, wherein the guiding element is comprised of a shape-memory material.

11. The prosthetic valve of claim 8, wherein the guiding element is formed from a wire.

12. The prosthetic valve of claim 8, wherein the leaflet comprises a polymeric material.

13. The prosthetic valve of claim 12, wherein the leaflet is formed from a composite material having more than one fluoropolymer layer.

14. The prosthetic valve of claim 13, wherein the guiding element is located between two fluoropolymer layers.

15. The prosthetic valve of claim 14, wherein the fluoropolymer layers comprise a plurality of pores.

16. The prosthetic valve of claim 15, wherein substantially all of the pores contain an elastomer.

17. The prosthetic valve of claim 16, wherein the elastomer comprises a fluoroelastomer.

18. The prosthetic valve of claim 16, wherein the elastomer comprises a TFE/PMVE copolymer.

19. The prosthetic valve of claim 17, wherein the fluoropolymer comprises PTFE.

20. The prosthetic valve of claim 19, wherein the PTFE is ePTFE.

21. The prosthetic valve of claim 1, wherein the guiding element defines a shape of one of a polygon, a square-sided oval, an undulating shape, a lemniscate, and an S-type shape.

22. A prosthetic valve comprising:
a frame;
at least one leaflet comprising a plurality of layers of film coupled together, each leaflet defining a leaflet base, a leaflet edge portion opposite the leaflet base, and a central portion between the leaflet base and the leaflet edge portion, wherein the leaflet is coupled to the frame along at least a portion of the leaflet base; and
a guiding element coupled between two of the plurality of layers of film that comprise the leaflet, the guiding element being located in the central portion and spaced apart from the frame.

23. The prosthetic valve of claim 22, wherein the guiding element is relatively more stiff compared to the plurality of layers of film.

24. The prosthetic valve of claim 23, wherein the guiding element defines a guiding element length and the leaflet defines a leaflet length extending from the leaflet base and the leaflet edge portion, the guiding element length being less than the leaflet length.

25. The prosthetic valve of claim 23, the leaflet further comprising a vertical axis and wherein the guiding element crosses the vertical axis.

26. The prosthetic valve of claim 23, the leaflet further comprising a vertical axis and wherein the guiding element is located substantially coincident with at least a portion of the vertical axis.

27. The prosthetic valve of claim 22, wherein the leaflet is operable to move between an open position and a closed position, wherein the guiding element is relatively more stiff compared to the plurality of layers of film, wherein a motion of the guiding element between the open position and a closed position carried by the leaflet follows a substantially planar pivoting surface.

28. The prosthetic valve of claim 27, wherein the motion of the central portion of the leaflet between the open position and a closed position substantially follows the guiding element.

29. The prosthetic valve of claim 22, wherein the guiding element is comprised of a shape-memory material.

30. The prosthetic valve of claim 22, wherein the guiding element is comprised of a metallic material.

31. The prosthetic valve of claim 22, wherein the guiding element is comprised of a shape-memory material.

32. The prosthetic valve of claim 22, wherein the guiding element is formed from a wire.

33. The prosthetic valve of claim 22, wherein the leaflet comprises a polymeric material.

34. The prosthetic valve of claim 33, wherein the leaflet is formed from a composite material having more than one fluoropolymer layer.

35. The prosthetic valve of claim 34, wherein the guiding element is located between two fluoropolymer layers.

36. The prosthetic valve of claim 35, wherein the fluoropolymer layers comprise a plurality of pores.

37. The prosthetic valve of claim 36, wherein substantially all of the pores contain an elastomer.

38. The prosthetic valve of claim 37, wherein the elastomer comprises a fluoroelastomer.

39. The prosthetic valve of claim 37, wherein the elastomer comprises a TFE/PMVE copolymer.

40. The prosthetic valve of claim 38, wherein the fluoropolymer comprises PTFE.

41. The prosthetic valve of claim 40, wherein the PTFE is ePTFE.

42. The prosthetic valve of claim 37, wherein the guiding element defines a shape of one of a polygon, a square-sided oval, an undulating shape, a lemniscate, and an S-type shape.

43. A prosthetic valve comprising:
a frame; and
at least one leaflet coupled to the frame, each leaflet comprising a plurality of layers of film coupled together, each leaflet defining a leaflet base, a leaflet edge portion opposite the leaflet base, and a central portion between the leaflet base and the leaflet edge portion, wherein the leaflet is coupled to the frame along at least a portion of the leaflet base, wherein each leaflet is pivotable between an open position and a closed position,
wherein the central portion has a greater stiffness than at least one of the leaflet edge portion and the leaflet base.

44. The prosthetic valve of claim 43, wherein an average stiffness varies throughout the central portion.

45. The prosthetic valve of claim 43, wherein an average stiffness varies throughout the central portion whereby the leaflet changes between a substantially concave shape and substantially convex shape as it pivots between the open position and the closed position.

46. The prosthetic valve of claim 43, wherein the stiffness of the central portion is increased relative to the leaflet base and the leaflet edge portion by at least one of an extra layer of film, a fiber, and a filament located between two of the plurality of layers of film that comprise the leaflet.

47. The prosthetic valve of claim 43, further comprising a guiding element coupled between two of the plurality of layers of film that comprise the leaflet, the guiding element being located in the central portion and spaced apart from the frame, the guiding element being relatively more stiff compared to the plurality of layers of film.

48. The prosthetic valve of claim 47, wherein the guiding element is selected from a list consisting of an extra layer of film, a sheet, a fiber, a filament, and a wire.

49. The prosthetic valve of claim 47, wherein the guiding element defines a guiding element length and the leaflet defines a leaflet length extending from the leaflet base and the leaflet edge portion, the guiding element length being less than the leaflet length.

50. The prosthetic valve of claim 47, the leaflet further comprising a vertical axis and wherein the guiding element crosses the vertical axis.

51. The prosthetic valve of claim 47, the leaflet further comprising a vertical axis and wherein the guiding element is located substantially coincident with at least a portion of the vertical axis.

52. The prosthetic valve of claim 47, wherein the guiding element is comprised of a shape-memory material.

53. The prosthetic valve of claim 47, wherein the guiding element is comprised of a metallic material.

54. The prosthetic valve of claim 47, wherein the guiding element is comprised of a shape-memory material.

55. The prosthetic valve of claim 47, wherein the guiding element is formed from a wire.

56. The prosthetic valve of claim 47, wherein the leaflet comprises a polymeric material.

57. The prosthetic valve of claim 56, wherein the leaflet is formed from a composite material having more than one fluoropolymer layer.

58. The prosthetic valve of claim 57, wherein the guiding element is located between two fluoropolymer layers.

59. The prosthetic valve of claim 58, wherein the fluoropolymer layers comprise a plurality of pores.

60. The prosthetic valve of claim 59, wherein substantially all of the pores contain an elastomer.

61. The prosthetic valve of claim 60, wherein the elastomer comprises a fluoroelastomer.

62. The prosthetic valve of claim 60, wherein the elastomer comprises a TFE/PMVE copolymer.

63. The prosthetic valve of claim 61, wherein the fluoropolymer comprises PTFE.

64. The prosthetic valve of claim 63, wherein the PTFE is ePTFE.

65. The prosthetic valve of claim 47, wherein the guiding element defines a shape of one of a polygon, a square-sided oval, an undulating shape, a lemniscate, and an S-type shape.

66. A leaflet for a prosthetic valve, comprising:
a plurality of layers of film coupled together configured in a form of the leaflet; and
one or more guiding elements coupled between two of the plurality of layers of film, the leaflet defining a leaflet edge portion and a leaflet base opposite from the leaflet edge portion and a central portion between the leaflet edge portion and the leaflet base, each guiding element being located in the central portion, the one or more guiding elements having a length which is aligned radiating away from but spaced apart from the leaflet base such that the leaflet pivots substantially from the leaflet base when the leaflet is deployed in the prosthetic valve and the prosthetic valve is operated so as to flex the leaflet.

67. The leaflet of claim 66 wherein the guiding element is relatively more stiff compared to the plurality of layers of film.

68. The leaflet of claim 67 wherein the one or more guiding elements are spaced a predetermined distance from a frame.
